(19) United States
(12) Patent Application Publication
(10) Pub. No.: US 2017/0231258 A1
(43) Pub. Date: Aug. 17, 2017

(54) RICE COOKING METHOD AND COOKED RICE

(71) Applicant: FLAT FIELD OPERATIONS CO., LTD., Osaka (JP)

(72) Inventor: Makoto HIRANO, Osaka (JP)

(21) Appl. No.: 15/428,324

(22) Filed: Feb. 9, 2017

(30) Foreign Application Priority Data
Feb. 16, 2016 (JP) 2016-027358

(51) Int. Cl. A23L 7/196; A23L 33/21; A61K 31/715; A23L 5/10

(52) U.S. Cl. CPC A23L 7/1965; A23L 5/13; A23L 33/21; A61K 31/715; A23V 2002/00

(57) ABSTRACT

A rice cooking method includes: a rice washing step of washing uncooked rice; a soaking step of soaking the uncooked rice, which has been washed in the rice washing step, in water; a rice cooking step of cooking the uncooked rice, which has been soaked in water in the soaking step, with the addition of water, indigestible dextrin as dietary fiber, and a seasoning liquid; and a steaming step of steaming rice that has been cooked in the rice cooking step. The seasoning liquid contains vinegar, fulvic acid, and mineral salt. The addition of the seasoning liquid containing vinegar, fulvic acid, and mineral salt in the rice cooking step can enhance the delicious taste, sweet taste, and water holding capacity of cooked rice.

### BACKGROUND OF THE INVENTION

The present invention relates to methods of cooking rice and to cooked rice.

Noodles as dietary-fiber functional food are known in the prior art. For example, Japanese Laid-open Patent Publication No. 2006-254901 discloses noodles in which indigestible dextrin, which is water-soluble dietary fiber, is added in order to achieve physiological effects such as blood sugar level rise suppression, an intestinal regulation effect, and lipid metabolism improvement. Indigestible dextrin is water-soluble and low-viscosity dietary fiber and can provide physiological effects such as blood sugar level rise suppression, an intestinal regulation effect, and lipid metabolism improvement.

### BRIEF SUMMARY OF THE INVENTION

When rice is cooked with indigestible dextrin added, the cooked rice is expected to have physiological effects such as blood sugar level rise suppression, an intestinal regulation effect, and lipid metabolism improvement. Further, it is desirable to enhance the delicious taste, sweet taste, and water holding capacity of cooked rice. Various methods of cooking rice have been proposed to enhance the delicious taste, sweet taste, and water holding capacity of cooked rice, but it is desirable to further increase the delicious taste, sweet taste, and water holding capacity of cooked rice.

The present invention has been made in view of the above circumstances. An object of the present invention is to provide: a rice cooking method that can enhance the delicious taste, sweet taste, and water holding capacity of cooked rice; and rice cooked by the method.

An aspect of the present invention provides a rice cooking method comprising: (claim 1 abbrev.)

Preferably, in the rice cooking step, (claim 2 abbrev.)

Another aspect of the present invention provides a rice cooking method comprising: (claim 3 abbrev.)

Preferably, in the rice cooking step, (claim 4 abbrev.)

A further aspect of the present invention provides cooked rice, (claim 5 abbrev.)

A further aspect of the present invention provides cooked rice (claim 6 abbrev.).

According to the present invention, the addition of the seasoning liquid, which contains vinegar, fulvic acid, and mineral salt, before cooking can enhance the delicious taste, sweet taste, and water holding capacity of cooked rice. Even when the amount of added water is increased by 10% compared to a conventional example, cooked rice can be un-soggy with rice grains standing well. Further, the yield in cooking can be improved close to 100%. Rice is cooked with the addition of the seasoning liquid containing vinegar, fulvic acid, and mineral salt. Thereby, even when the cooked rice is frozen and thereafter thawed and heated, it can have a sweet and delicious taste like freshly cooked rice. Further, the moisture in the cooked rice can be retained even after the heating.

### BRIEF DESCRIPTION OF THE DRAWINGS

FIG. 1 is a flow chart showing a rice cooking method according to one embodiment of the present invention.

### DETAILED DESCRIPTION OF THE INVENTION

Referring now to the accompanying drawing, rice cooking methods and cooked rice according to embodiments of the present invention are described.

### First Embodiment

In a rice cooking method according to a first embodiment of the present invention, indigestible dextrin as dietary fiber and a fulvic acid mineral seasoning liquid are added to uncooked rice before the rice is cooked. The fulvic acid mineral seasoning liquid (hereinafter referred to as “seasoning liquid”) contains vinegar, fulvic acid, and mineral salt. The indigestible dextrin is water-soluble and low-viscosity dietary fiber and can provide physiological effects such as blood sugar level rise suppression, an intestinal regulation effect, and lipid metabolism improvement. The mineral salt in the seasoning liquid contains at least any one of calcium, potassium, and magnesium.

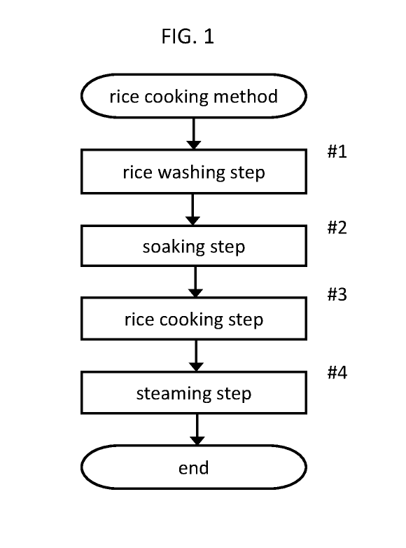

FIG. 1 shows a flow chart of the rice cooking method of this embodiment. The rice cooking method of this embodiment includes a rice washing step S1, a soaking step S2, a rice cooking step 3, and a steaming step S4. In the rice washing step S1, uncooked rice is rinsed with water and washed. Pre-washed rice is used here as the uncooked rice. The uncooked rice is washed three or four times and then rinsed with water three times. The rice washing should be performed quickly because water is absorbed quickly three or more minutes after the start of rinsing.

In the soaking step S2, the uncooked rice, which has been washed in the rice washing step S1, is soaked in water so as to absorb water. The temperature of water in which the uncooked rice is soaked is adjusted to around 20 degrees C. The length of soaking time is approximately 60 minutes. When uncooked rice is soaked under these conditions, the weight of soaked rice, which has absorbed water, is approximately 1.3 times the weight of uncooked rice before soaking. It is desirable that the weight of soaked rice is about 1.3 times the weight of uncooked rice before soaking.

In the rice cooking step S3, water, indigestible dextrin as dietary fiber, and the seasoning liquid are added to the uncooked rice, which has been soaked in water in the soaking step S2, and then the rice is cooked. At that time, 110 to 140 weight % of water is added to 100 weight % of uncooked rice that has been soaked in water in the soaking step. Further, 3 to 15 weight % of indigestible dextrin and 0.08 to 0.15 weight % of the seasoning liquid are added to 100 weight % of the added water. In one example, the seasoning liquid contains 78% water, 20% vinegar, 1.5% fulvic acid, and 0.5% mineral salt. The blend ratio can be changed appropriately. It is to be noted that water is drained once after the soaking step and then water for cooking is added. The rice is cooked on low heat (at around 40 degrees C.) for the first 4 minutes and then cooked on high heat to be boiled (ata temperature of 98 degrees C. or higher) for 8 minutes. After the temperature is maintained at 98 degrees C. for 8 minutes from the start of boiling, the heat is shut off. Thereby, rice is cooked.

In the steaming step S4, the rice that has been cooked in the rice cooking step S3 is steamed. For steaming rice, the temperature is maintained at 98 degrees C. for 20 minutes. If the temperature falls below 98 degrees C., it is adjusted by reheating. Accordingly, rice cooking is completed. Cooked rice obtained by the rice cooking method of this embodiment is rice cooked with indigestible dextrin and the seasoning liquid added.

A yield ratio (herein after abbreviate to yield) been investigated according to the cooking method of this embodiment and a conventional cooking method. The results are shown in Table 1. The yield is an ingredient percentage of cooked rice weight relative to the total weight before cooking. In Table 1, the weight of uncooked rice is the weight of rice that has been soaked in water in the soaking step while the weight of water, the seasoning liquid, or indigestible dextrin is the weight of the each ingredient added in the rice cooking step. “Total” indicates the total weight of the ingredients before cooking. “Cooked weight” indicates the weight of rice that has been cooked.

TABLE 1

| | |
|---|---|
| Conventional | |
| example | Example 1 |

| | | | |
|---|---|---|---|
| Uncooked rice | 1 | kg | 1 kg |

| | | |
|---|---|---|
| Water | 1.2 kg | 1.32 kg |
| | (120 weight % | (132 weight % |
| | relative to | relative to |
| | uncooked rice) | uncooked rice) |

| | | | |
|---|---|---|---|
| Fulvic acid | | | 1.65 g |
| mineral seasoning liquid | | | (0.125 weight % |
| | | | relative to water) |
| Indigestible dextrin | | | 66 g |
| | | | (5 weight % |
| | | | relative to water) |
| Total | 2.2 | kg | 2.387 kg |
| Cooked weight | 1.815 | kg | 2.315 kg |

| | | |
|---|---|---|
| Yield | 82.5% | 97% |

The conventional example is different from this embodiment in that the seasoning liquid and indigestible dextrin are not added. In the conventional example, 1 kilogram (kg) of uncooked rice that has been soaked in water is used, and 1.2 kg (120 weight % relative to the uncooked rice that has been soaked in water) of water is added to the uncooked rice. In the conventional example, while the total weight of ingredients before cooking is 2.2 kg, the weight of cooked rice is 1.815 kg. Thus, the yield is 82.5%.

Example 1 is a rice cooking method according to this embodiment. In this example, 1 kg of uncooked rice that has been soaked in water in the soaking step is used. In the rice cooking step, 1.32 kg (132 weight % relative to the uncooked rice that has been soaked in water) of water is added, and further, 1.65 gram (g) (0.125 weight % relative to the added water) of the seasoning liquid and 66 g (5 weight % relative to the added water) of indigestible dextrin are added. In other words, in Example 1, the amount of water added in the rice cooking step is increased by approximately 10% compared to the conventional example. Further, the seasoning liquid and indigestible dextrin are added unlike the conventional example. In Example 1, while the total weight of ingredients before cooking is 2.387 kg, the weight of cooked rice is 2.315 kg. Thus, the yield is 97%.

In Example 1 described above, the yield in cooking is improved close to 100%. This effect can be achieved by the addition of the seasoning liquid before cooking, which can prevent water vapor from escaping. By improving the yield, production costs can be reduced.

It has been determined that rice cooked according to Example 1 has grains standing well and is un-soggy. Rice cooked according to Example 1 has been tasted as well as rice cooked according to the conventional example. As a result, it has been determined that Example 1 can bring out the natural sweetness and flavor of rice sufficiently as compared to the conventional example. This effect can be achieved by the addition of the seasoning liquid before cooking.

Rice cooked according to the conventional example or Example 1 was covered with plastic wrap and kept in a freezer for 5 days. Thereafter, the rice was thawed, heated, and then tasted. As a result, it has been determined that the rice cooked according to the conventional example smells of plastic wrap and begins to dry right after heating. In contrast, it has been determined that the rice cooked according to Example 1 does not smell of plastic wrap and has a sweet and delicious taste like freshly cooked rice, and that time between heating and drying can be lengthened as compared to the conventional example. This effect can be achieved by the addition of the seasoning liquid before cooking.

The content of dietary fiber in rice cooked according to Example 1 has been investigated. As a result, 5.6 g of dietary fiber is contained per 100 g of cooked rice. The content of dietary fiber is higher than that in commonly cooked rice (approximately 0.3 g per 100 g of cooked rice). More particularly, dissolution of indigestible dextrin, which is water-soluble dietary fiber, is suppressed (the dissolution rate is lowered). This effect can be achieved by the addition of indigestible dextrin before cooking.

In another example, 132 g (10 weight % relative to added water) of indigestible dextrin is added instead of 66 g of indigestible dextrin. This example can also have an effect similar to the above. In this example, 7.2 g of dietary fiber is contained per 100 g of cooked rice.

According to the rice cooking method of this embodiment, the addition of the seasoning liquid before cooking can enhance the delicious taste, sweet taste, and water holding capacity of cooked rice. Even when the amount of added water is increased by 10% compared to the conventional example, cooked rice can be un-soggy with rice grains standing well. Further, the cooking yield can be improved close to 100%. Even when rice cooked according to the rice cooking method of this embodiment is frozen and thereafter thawed and heated, it can have a sweet and delicious taste like freshly cooked rice. Further, the moisture in the cooked rice can be retained even after the heating. Moreover, the indigestible dextrin contained therein can provide physiological effects such as blood sugar level rise suppression, an intestinal regulation effect, and lipid metabolism improvement.

### Second Embodiment

In a rice cooking method according to a second embodiment of the present invention, a seasoning liquid is added to uncooked rice before the rice is cooked. The rice cooking method of this embodiment has a rice cooking step different from that in the above described first embodiment. In this rice cooking step, water and the seasoning liquid are added to uncooked rice, which has been soaked in water in a soaking step, and then the rice is cooked. The amount of added water is 110 to 140 weight % relative to 100 weight % of uncooked rice that has been soaked in water in the soaking step. Further, 0.08 to 0.15 weight % of the seasoning liquid is added to 100 weight % of the added water. Like the above first embodiment, the rice is cooked on low heat (at around 40 degrees C.) for the first 4 minutes and then cooked on high heat to be boiled (at a temperature of 98 degrees C. or higher) for 8 minutes. After the temperature is maintained at 98 degrees C. for 8 minutes from the start of boiling, the heat is shut off. The other steps are similar to those in the above first embodiment.

The yield have been investigated according to the rice cooking method of this embodiment (Examples 2, 3, and 4) and a conventional rice cooking method. The results are shown in Table 2. In Table 2, the weight of rice is the weight of rice that has been soaked in water in the soaking step while the weight of water or the seasoning liquid is the weight of the each ingredient added in the rice cooking step. “Total” indicates the total weight of the ingredients before cooking. “Cooked” indicates the weight of rice that has been cooked.

TABLE 2

| | | | Example 3 | Example 4 |
|---|---|---|---|---|
| | | Example 2 | seasoning liquid | seasoning liquid |
| | | seasoning liquid | diluted by a factor | diluted by a factor |
| | Conventional | diluted by a | of 1000, and water | of 1000, and water |
| | Example | factor of 1000 | increased by about 5% | increased by about 10% |
| Rice (g) | 300 | 300 | 300 | 300 |
| Water (g) | 360 | 360 | 380 | 400 |
| | (120 weight % | (120 weight % | (127 weight % | (133 weight % |
| | relative to rice) | relative to rice) | relative to rice) | relative to rice) |
| Fulvic acid | | 0.36 | 0.36 | 0.36 |
| mineral seasoning | | (0.1 weight % | (0.095 weight % | (0.09 weight % |
| liquid (g) | | relative to water) | relative to water) | relative to water) |
| Total (g) | 660 | 660 | 680 | 700 |
| Cooked (g) | 545 | 643 | 663 | 679 |
| Yield | 82.50% | 97.40% | 97.50% | 97% |

The conventional example is different from this embodiment in that the seasoning liquid is not added. In the conventional example, 300 g of uncooked rice that has been soaked in water is used, and 360 g (120 weight % relative to the uncooked rice that has been soaked in water) of water is added to the uncooked rice. In the conventional example, while the total weight of ingredients before cooking is 660 g, the weight of cooked rice is 545 g. Thus, the yield is 82.50%.

In Example 2, 300 g of uncooked rice that has been soaked in water in the soaking step is used. In the rice cooking step, 360 g of the seasoning liquid diluted by a factor of approximately 1000 with water is added to the uncooked rice. More particularly, in the rice cooking step of Example 2, 360 g (120 weight % relative to the uncooked rice that has been soaked in water in the soaking step) of water is added along with 0.36 g (0.1 weight % relative to the added water) of the seasoning liquid. In other words, in Example 2, the weight of water added in the rice cooking step is equal to that in the conventional example, and further, the seasoning liquid is added. In Example 2, while the total weight of ingredients before cooking is 660 g, the weight of cooked rice is 643 g. Thus, the yield is 97. 40%.

In Example 3, 300 g of uncooked rice that has been soaked in water in the soaking step is used. In the rice cooking step, 360 g of the seasoning liquid diluted by a factor of approximately 1000 with water is added. Further, the amount of added water is increased by approximately 5% compared to the conventional example. More particularly, in the rice cooking step of Example 3, 380 g (127 weight % relative to the uncooked rice that has been soaked in water in the soaking step) of water is added along with 0.36 g (0.095 weight % relative to the added water) of the seasoning liquid. In other words, in Example 3, the weight of water added in the rice cooking step is increased by approximately 5% compared to the conventional example while the weight of the seasoning liquid added in the step is equal to that in Example 2. In Example 3, while the total weight of ingredients before cooking is 680 g, the weight of cooked rice is 663 g. Thus, the yield is 97.50%.

In Example 4, 300 g of uncooked rice that has been soaked in water in the soaking step is used. In the rice cooking step, 360 g of the seasoning liquid diluted by a factor of approximately 1000 with water is added. Further, the amount of added water is increased by approximately 10% compared to the conventional example. More particularly, in the rice cooking step of Example 4, 400 g (133 weight % relative to the uncooked rice that has been soaked in water in the soaking step) of water is added along with 0.36 g (0.09 weight % relative to the added water) of the seasoning liquid. In other words, in Example 4, the weight of water added in the rice cooking step is increased by approximately 10% compared to the conventional example while the weight of the seasoning liquid added in the step is equal to that in Example 2. In Example 4, while the total weight of ingredients before cooking is 700 g, the weight of cooked rice is 679 g. Thus, the yield is 97%.

As described above, while the cooking yield in the conventional example is 82.50%, the cooking yields in Examples 2, 3, and 4 are 97.40%, 97.50%, and 97%, respectively. Thus, in any of Examples 2, 3, and 4, the cooking yield is improved close to 100%. This effect can be achieved by the addition of the seasoning liquid before cooking in each of Examples 2, 3, and 4, which can prevent water vapor from escaping.

It has been determined that rice cooked according to the rice cooking method of Example 2, 3, or 4 is un-soggy with rice grains standing well. Rice cooked according to the method of Example 2, 3, or 4 has been tasted as well as rice cooked according to the method of the conventional example. As a result, it has been determined that the methods of Examples 2, 3, and 4 can bring out the natural sweetness and flavor of rice sufficiently as compared to the method of the conventional example. This effect can be achieved by the addition of the seasoning liquid before cooking in Examples 2, 3, and 4.

Rice cooked according to the conventional example or Example 2, 3, or 4 was covered with plastic wrap and kept in a freezer for 5 days. Thereafter, the rice was thawed, heated, and then tasted. As a result, it has been determined that the rice cooked according to the conventional example smells of plastic wrap and begins to dry right after heating. In contrast, it has been determined that the rice cooked according to Example 2, 3, or 4 does not smell of plastic wrap and has a sweet and delicious taste like freshly cooked rice, and that time between heating and drying can be lengthened as compared to the conventional example. This effect can be achieved by the addition of the seasoning liquid before cooking.

According to the rice cooking method of this embodiment, the addition of the seasoning liquid before cooking can enhance the delicious taste, sweet taste, and water holding capacity of cooked rice. Even when the amount of added water is increased by 10% compared to the conventional example, cooked rice can be un-soggy with rice grains standing well. Further, the cooking yield can be improved close to 100%. Rice cooked according to the rice cooking method of this embodiment is rice cooked with the addition of the seasoning liquid. Thereby, even when the cooked rice is frozen and thereafter thawed and heated, it can have a sweet and delicious taste like freshly cooked rice. Further, the moisture in the cooked rice can be retained even after the heating.

The prevent invention is not limited to the above embodiments and various modifications may be made. For example, in place of pre-washed rice, polished rice may be used as uncooked rice. The number of times uncooked rice is rinsed with water in the rice washing step may be changed. Further, in addition to the seasoning liquid and the indigestible dextrin, another additive may be added to the extent that the qualities are not affected.

This application is based upon and claims the benefit of priority from Japanese Patent Application No. 2016-027358 filed on Feb. 16, 2016, the entire contents of which are incorporated herein by reference.

1. A rice cooking method comprising:
a rice washing step of washing uncooked rice;
a soaking step of soaking the uncooked rice, which has been washed in the rice washing step, in water;
a rice cooking step of cooking the uncooked rice, which has been soaked in water in the soaking step, with addition of water, indigestible dextrin as dietary fiber, and a seasoning liquid; and
a steaming step of steaming rice that has been cooked in the rice cooking step,
wherein the seasoning liquid contains vinegar, fulvic acid, and mineral salt.

2. The rice cooking method according to claim 1, wherein in the rice cooking step, 110 to 140 weight % of water is added to 100 weight % of uncooked rice, which has been soaked in water in the soaking step, and 3 to 15 weight % of indigestible dextrin and 0.08 to 0.15 weight % of the seasoning liquid are added to 100 weight % of the water added.

3. A rice cooking method comprising:
a rice washing step of washing uncooked rice;
a soaking step of soaking the uncooked rice, which has been washed in the rice washing step, in water;
a rice cooking step of cooking the uncooked rice, which has been soaked in water in the soaking step, with addition of water and a seasoning liquid; and
a steaming step of steaming rice that has been cooked in the rice cooking step,
wherein the seasoning liquid contains vinegar, fulvic acid, and mineral salt.

4. The rice cooking method according to claim 3, wherein in the rice cooking step, 110 to 140 weight % of water is added to 100 weight % of uncooked rice, which has been soaked in water in the soaking step, and 0.08 to 0.15 weight % of the seasoning liquid is added to 100 weight % of the water added.

5. Cooked rice comprising rice cooked with addition of a seasoning liquid containing vinegar, fulvic acid, and mineral salt.

6. Cooked rice comprising rice cooked with addition of indigestible dextrin as dietary fiber and a seasoning liquid containing vinegar, fulvic acid, and mineral salt.
